(19) United States
(12) Patent Application Publication
(10) Pub. No.: US 2023/0145041 A1
(43) Pub. Date: May 11, 2023

(54) APPARATUS AND METHOD FOR GENE AMPLIFICATION

(71) Applicants: SAMSUNG ELECTRONICS CO., LTD., Suwon-si (KR); SEOUL NATIONAL UNIVERSITY R&DB FOUNDATION, Seoul (KR)

(72) Inventors: Won Jong JUNG, Seoul (KR); Ho-Young Kim, Seoul (KR); Hyeong Seok Jang, Seoul (KR); Kak Namkoong, Seoul (KR); Tae Jeong Kim, Seoul (KR); Jae Hong Lee, Seongnam-si (KR); Sohyun Jung, Anyang-si (KR)

(73) Assignees: SAMSUNG ELECTRONICS CO., LTD., Suwon-si (KR); SEOUL NATIONAL UNIVERSITY R&DB FOUNDATION, Seoul (KR)

(21) Appl. No.: 17/684,884

(22) Filed: Mar. 2, 2022

(30) Foreign Application Priority Data
Nov. 8, 2021 (KR) 10-2021-0151948

(51) Int. Cl. B01L 3/00; C12Q 1/6876; C12Q 1/6844; G01N 21/47

(52) U.S. Cl. CPC B01L 3/502715; C12Q 1/6876; C12Q 1/6844; G01N 21/47; B01L 2300/0654; B01L 2300/0819; B01L 2300/161; B01L 2400/0406

(57) ABSTRACT

The present disclosure relates to an apparatus and method for gene amplification. The apparatus for gene amplification may include: an upper main body comprising a first inlet to receive a sealing solution, a second inlet to receive a sample solution, and an upper passage that allows the sample solution and the sealing solution to move by capillary action; a lower main body disposed to oppose the upper main body, and having a lower passage through which the sealing solution moves by capillary action after being injected from the first inlet of the upper main body; a gene amplification chip configured to be inserted between the upper main body and the lower main body; and a porous medium configured to be inserted between the upper main body and the lower main body.

### CROSS-REFERENCE TO RELATED APPLICATION(S)

This application claims priority from Korean Patent Application No. 10-2021-0151948, filed on Nov. 8, 2021, in the Korean Intellectual Property Office, the disclosure of which is incorporated herein by reference in its entirety.

### BACKGROUND

### 1. Field

Apparatuses and methods consistent with example embodiments relate to amplifying a gene extracted from a biological sample to identify genetic mutations and various infections.

### 2. Description of the Related Art

Clinical or environmental samples are analyzed by a series of biochemical, chemical, and mechanical treatment processes. Recently, there has been considerably increasing interest in developing techniques for diagnosis or monitoring of biological samples. Molecular diagnosis based on nucleic acid amplification techniques has excellent accuracy and sensitivity, and thus is increasingly used in various applications, ranging from diagnosis of infectious diseases or cancer to pharmacogenomics, development of new drugs, and the like. Microfluidic devices are widely used to analyze samples in a simple and accurate manner according to various purposes.

### SUMMARY

According to an aspect of the present disclosure, there is provided an apparatus for gene amplification, the apparatus including: an upper main body comprising a first inlet to receive a sealing solution, a second inlet to receive a sample solution, and an upper passage that allows the sample solution and the sealing solution to move by capillary action; a lower main body disposed to oppose the upper main body, and having a lower passage through which the sealing solution moves by capillary action after being injected from the first inlet of the upper main body; a gene amplification chip configured to be inserted between the upper main body and the lower main body; and a porous medium configured to be inserted between the upper main body and the lower main body.

The upper passage may include a first injection path for guiding the sample solution and the sealing solution toward the gene amplification chip, a first main flow path disposed on an upper portion of the gene amplification chip, and a first discharge path for guiding the sample solution and the sealing solution toward the porous medium. The lower passage may include a second injection path for guiding the sealing solution toward the gene amplification chip, a second main flow path disposed on a lower portion of the gene amplification chip, and a second discharge path for guiding the sealing solution toward the porous medium.

The first main flow path may be inclined from the first injection path toward the first discharge path, or the second main flow path may be inclined from the second injection path toward the second discharge path.

At least one of the first main flow path and the second main flow path may have an inclination angle of 0° to 35°.

At least one of the first injection path, the first discharge path, the second injection path, and the second discharge path may have a width which is not constant in a flow direction of the sample solution or the sealing solution.

A width of the first injection path and a width of the second injection path may linearly decrease in the flow direction of the sample solution or the sealing solution.

A width of the first discharge path and a width of the second discharge path may linearly increase in the flow direction of the sample solution or the sealing solution.

A width of the first injection path, a width of the second injection path, a width of the first discharge path, and a width of the second discharge path may be in a range of 1 μm to 5 mm. A width of the first main flow path and a width of the second main flow path may be in a range of 1 μm to 10 cm.

The upper main body may further include an auxiliary channel which is provided on both sides of the upper passage, and which allows the sealing solution to move by capillary action through the auxiliary channel.

The auxiliary channel may be stepped with respect to the upper passage.

The upper passage and the lower passage may include a hydrophilic material having a contact angle of 90° or less with respect to water.

The sealing solution may be a non-polar solution that is not mixed with the sample solution.

The porous medium may include a hydrophilic material, and may have a plurality of pores or a plurality of pin type microstructures.

A diameter of each of the plurality of pores or each of the plurality of pin type microstructures may be in a range of 0.001 μm to 100 μm, and may be smaller than a width of the upper passage and a width of the lower passage. A distance between the plurality of pores or a distance between the plurality of pin type microstructures may be in a range of 0.001 μm to 100 μm.

A diameter of the first inlet may be greater than a diameter of the second inlet.

The diameter of the first inlet may be greater than or equal to a width of an injection path of the upper passage; and the diameter of the second inlet may be in a range of 0.1 μm to 4500 μm.

The upper main body may further include an air pressure maintenance hole disposed on an upper portion of the porous medium.

The gene amplification chip may include a substrate, and an array of through holes which pass through the substrate in a direction from an upper surface to a lower surface of the substrate, and in which a gene amplification reaction occurs.

The gene amplification chip may include a photothermal film disposed on at least one of the upper surface and the lower surface of the substrate, and a partition wall of the respective through holes, and generating heat by using received light.

According to another aspect of the present disclosure, there is provided an apparatus for detecting a microfluid, the apparatus including: a gene amplifier; an optical unit including a light emitter and a light detector to emit light onto a sample solution and to measure an optical signal scattered or reflected from the sample solution, while a gene amplification reaction is performed in a gene amplification chip of the gene amplifier, or after the gene amplification reaction is complete; and a processor configured to detect an amplified gene by analyzing the optical signal, wherein the gene amplifier may include: an upper main body comprising a first inlet to receive a sealing solution, a second inlet to receive the sample solution, and an upper passage that allows the sample solution and the sealing solution to move by capillary action; a lower main body disposed to oppose the upper main body, and having a lower passage through which the sealing solution moves by capillary action after being injected from the first inlet of the upper main body; the gene amplification chip configured to be inserted between the upper main body and the lower main body; and a porous medium configured to be inserted between the upper main body and the lower main body.

### BRIEF DESCRIPTION OF THE DRAWINGS

The above and/or other aspects will be more apparent by describing certain example embodiments, with reference to the accompanying drawings, in which:

FIG. 1 is a block diagram illustrating an apparatus for gene amplification according to an embodiment of the present disclosure;

FIG. 2A is a front view of an apparatus for gene amplification according to an embodiment of the present disclosure;

FIG. 2B is a plan view of an apparatus for gene amplification according to an embodiment of the present disclosure;

FIG. 3A is a plan view of an apparatus for gene amplification according to another embodiment of the present disclosure;

FIGS. 3B and 3C are diagrams illustrating a structure and an effect of an auxiliary channel;

FIG. 4A is a diagram illustrating a gene amplification chip according to an embodiment of the present disclosure;

FIG. 4B is a diagram illustrating a side surface of a gene amplification chip, on which a photothermal film is deposited;

FIGS. 5 to 8 are block diagrams illustrating an apparatus for detecting a microfluid according to embodiments of the present disclosure; and

FIG. 9 is a flowchart illustrating a method of gene amplification according to an embodiment of the present disclosure.

### DETAILED DESCRIPTION

Example embodiments are described in greater detail below with reference to the accompanying drawings.

In the following description, like drawing reference numerals are used for like elements, even in different drawings. The matters defined in the description, such as detailed construction and elements, are provided to assist in a comprehensive understanding of the example embodiments. However, it is apparent that the example embodiments can be practiced without those specifically defined matters. Also, well-known functions or constructions are not described in detail since they would obscure the description with unnecessary detail.

It will be understood that, although the terms first, second, etc. may be used herein to describe various elements, these elements should not be limited by these terms. These terms are only used to distinguish one element from another. Any references to singular may include plural unless expressly stated otherwise. In addition, unless explicitly described to the contrary, an expression such as “comprising” or “including” will be understood to imply the inclusion of stated elements but not the exclusion of any other elements. Also, the terms, such as ‘unit’ or ‘module’, etc., should be understood as a unit that performs at least one function or operation and that may be embodied as hardware, software, or a combination thereof. Expressions such as “at least one of,” when preceding a list of elements, modify the entire list of elements and do not modify the individual elements of the list. For example, the expression, “at least one of a, b, and c,” should be understood as including only a, only b, only c, both a and b, both a and c, both b and c, all of a, b, and c, or any variations of the aforementioned examples.

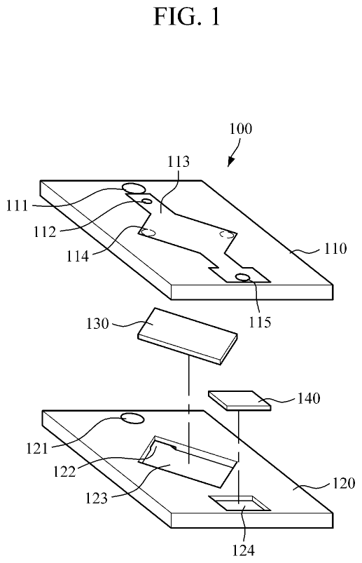

FIG. 1 is a block diagram illustrating an apparatus 100 for gene amplification according to an embodiment of the present disclosure. Referring to FIG. 1, the apparatus 100 for gene amplification includes an upper main body 110, a lower main body 120, a gene amplification chip 130, and a porous medium 140. The apparatus 100 may be also referred to as a gene amplifier.

The upper main body 110 includes a first inlet 111, a second inlet 112, an upper passage 113, a gene amplification chip fixing column 114, and an air pressure maintenance hole 115.

The first inlet 111 may be an inlet into which a sealing solution is injected, and the second inlet 112 may be an inlet into which a sample solution is injected.

The sealing solution may be a non-polar solution that is not mixed with the sample solution. In particular, the sealing solution may be oil, but is not limited thereto. When a gene amplification reaction occurs with the sample solution being loaded into the gene amplification chip 130, if an upper surface and a lower surface of the gene amplification chip 130 are in contact with a gas, the sample solution may be evaporated and lost rapidly during the gene amplification reaction. In this case, the sealing solution may be coated on the upper surface, the lower surface, and the like of the gene amplification chip 130, thereby preventing loss of the loaded sample solution.

The sample solution may be bio-fluids, including at least one of respiratory secretions, blood, urine, perspiration, tears, saliva, etc., or a swab sample of the upper respiratory tract, or a solution of the bio-fluid or the swab sample dispersed in other medium. In this case, the other medium may include water, saline solution, alcohol, phosphate buffered saline solution, vital transport media, etc., but is not limited thereto. A volume of the sample may be in a range of 1 μL to 1000 μL, but is not limited thereto.

The sample solution may contain microbes. The microbes may include a duplex of one or more of ribonucleic acid (RNA) virus, deoxyribonucleic acid (DNA) virus, peptide nucleic acid (PNA) virus, and locked nucleic acid (LNA) virus, bacteria, pathogen, germ, virus, oligopeptide, protein, toxin, etc., but the microbes are not limited thereto.

The microbes may contain genes. For example, the genes may include a duplex of one or more of ribonucleic acid (RNA), deoxyribonucleic acid (DNA), peptide nucleic acid (PNA), and locked nucleic acid (LNA), but the genes are not limited thereto.

While FIG. 1 illustrates the first inlet 111 and the second inlet 112 as having a circular shape, but the shape is not limited thereto, and the first inlet 111 and the second inlet 112 may have a polygonal shape, such as square, pentagon, and the like.

The gene amplification chip fixing column 114 is disposed on an upper portion of the gene amplification chip 130, to fix the gene amplification chip 130 so that the gene amplification chip 130, inserted between the upper main body 110 and the lower main body 120, may not be separated to the outside.

The air pressure maintenance hole 115 may be disposed on an upper portion of the porous medium 140. The air pressure maintenance hole 115 may serve to maintain gas pressure in the upper passage 113, the lower passage 122, and the porous medium 140 at atmospheric pressure. In this case, a diameter of the air pressure maintenance hole 115 may be in a range of 10 μm to 5 mm, but is not limited thereto.

Although not illustrated in FIG. 1 for convenience of explanation, the upper main body 110 may further include an insertion groove of the gene amplification chip and an insertion groove of the porous medium. The respective insertion grooves may be formed at positions corresponding to an insertion groove 123 of the gene amplification chip and an insertion groove 124 of the porous medium in the lower main body 120.

The lower main body 120 may include a first inlet connector 121, a lower passage 122, the insertion groove 123 of the gene amplification chip and the insertion groove 124 of the porous medium.

The first inlet connector 121 may be formed at a position corresponding to the first inlet 111 of the upper main body 110, as illustrated in FIG. 1. The sealing solution injected from the first inlet 111 of the upper main body 110 may be injected into the lower main body 120 through the first inlet connector 121 of the lower main body 120.

The gene amplification chip 130 may be inserted between the insertion groove 123 of the gene amplification chip in the lower main body 120 and the insertion groove of the gene amplification chip in the upper main body 110. The porous medium 140 may be inserted between the insertion groove 124 of the porous medium in the lower main body 120 and the insertion groove of the porous medium in the upper main body 110.

Although not illustrated in FIG. 1 for convenience of explanation, the lower main body 120 may further include a gene amplification chip fixing column, in which case the gene amplification chip fixing column of the lower main body 120 may be disposed at a position corresponding to the gene amplification chip fixing column 114 of the upper main body 110.

The upper passage 113 and the lower passage 122 may be made of an inorganic matter, such as silicon (Si), glass, polymer, metal, ceramic, graphite, etc., acrylic material, polyethylene terephthalate (PET), polycarbonate, polystylene, and polypropylene, but is not limited thereto.

The sample solution may be loaded into the gene amplification chip 130 through the upper passage 112, and the sample solution and the sealing solution may move toward the porous medium 140 through the upper passage 113 and/or the lower passage 122. In this case, the injected sample solution and sealing solution may move by capillary action.

For example, the injected sample solution may be loaded into the gene amplification chip 130 by capillary action through the upper passage 112, in which case the sample solution, not loaded into the gene amplification chip 130, may move by capillary action toward the porous medium 140 through the upper passage 113. The sealing solution injected thereafter may move by capillary action toward the gene amplification chip 130 and the porous medium 140 through the upper passage 113 and the lower passage 122. In this case, the sealing solution may be filled in all of the upper passage 113, the lower passage 112, the upper surface and lower surface of the gene amplification chip 130, and the porous medium 140, thereby preventing the sample solution, loaded into the gene amplification chip 130, from being in contact with a gas.

A material or a structure for pre-treatment of the sample solution may be provided inside or outside of the upper passage 113, in which case the sample solution may be pre-treated before being loaded into the gene amplification chip 130 through the upper passage 113. For example, a pre-treatment process, such as heating, chemical treatment, treatment with magnetic beads, solid phase extraction, treatment with ultrasonic waves, etc., may be performed. The upper passage 113 may include a filter for passing only a fluid while blocking fine particles in the pre-treated sample. The filter may be formed in the shape of a single layer or multilayer film having microholes, and may block fine particles of a desired size according to the size of the holes. The filter may be made of, for example, silicon, polyvinylidene fluoride (PVDF), polyethersulfone, polycarbonate, glass fiber, Polypropylene, Cellulose, Mixed cellulose esters, Polytetrafluoroethylene (PTFE), Polyethylene Terephthalate, Polyvinyl chloride (PVC), Nylon, Phosphocellulose, Diethylaminoethyl cellulose (DEAE), and the like, but is not limited thereto. The holes may have various shapes, e.g., a circular shape, a rectangular shape, a slit shape, an irregular shape due to glass fiber, and the like.

In addition, the upper passage 113 may include a field effect transistor (FET), a silicon (Si) photonic structure, a 2D micro/nano material/structure, and the like. Further, the upper passage 113 may include a structure having optical or electrical heating properties for controlling temperature of the sample.

The upper passage 113 may contain reactants for each gene to be amplified. In this case, the gene to be amplified may be, for example, a duplex of one or more of ribonucleic acid (RNA), deoxyribonucleic acid (DNA), peptide nucleic acid (PNA), and locked nucleic acid (LNA), oligopeptide, protein, toxin, and the like. The reactants for each gene may include, for example, reverse transcriptase, polymerase, ligase, peroxidase, primer, probe, etc., but is not limited thereto. The primer may include oligonucleotide, for example, target specific single strand oligonucleotide. Further, the probe may include oligonucleotide, for example, target specific single strand oligonucleotide, a fluorescent material, quencher, and the like. The probe may exhibit a characteristic fluorescence signal by interacting with a specific target material in a solution, in which different types of materials are dissolved. Such characteristic signal may be tracked, detected, and processed for a predetermined period of time by an optical unit and/or a processor of the apparatus for gene amplification, for use in detecting the amplified gene.

The upper passage 113 and/or the lower passage 122 may be made of a material and a structure for facilitating capillary action.

For example, the upper passage 113 and/or the lower passage 122 may be made of a hydrophilic material having a contact angle of 90° or less with respect to water. For example, the upper passage 113 and/or the lower passage 122 may be made of a material having a contact angle of 10° or less with respect to water, but the material is not limited thereto.

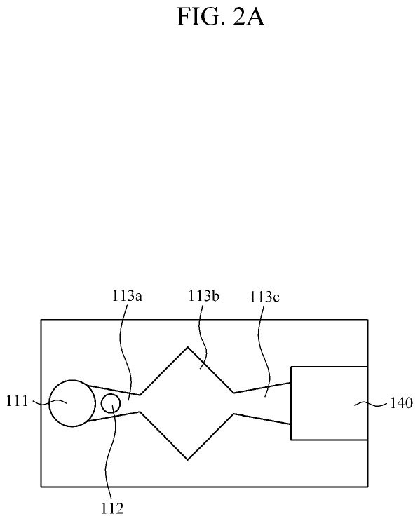

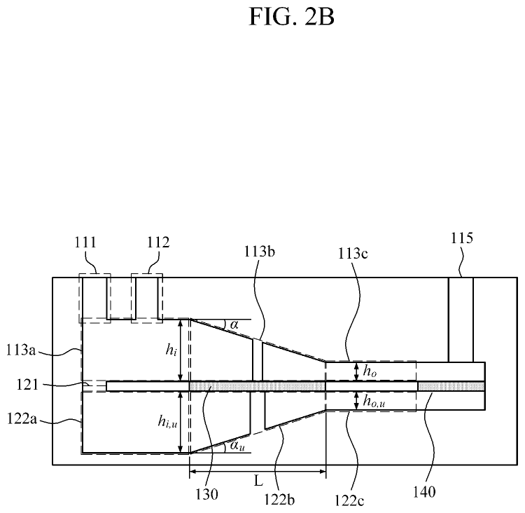

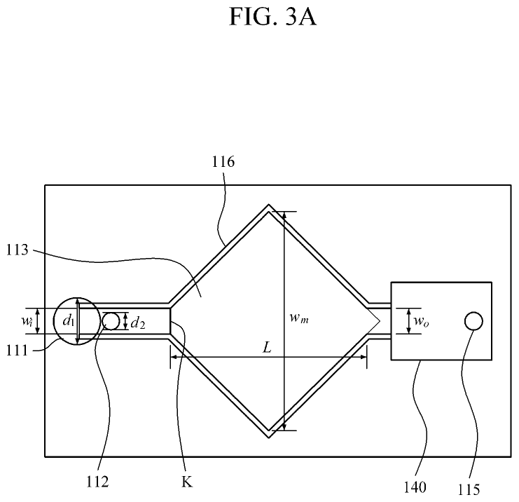

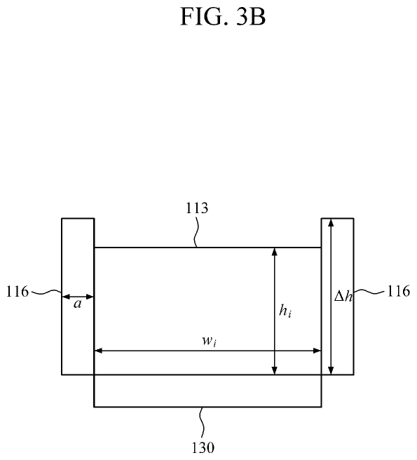

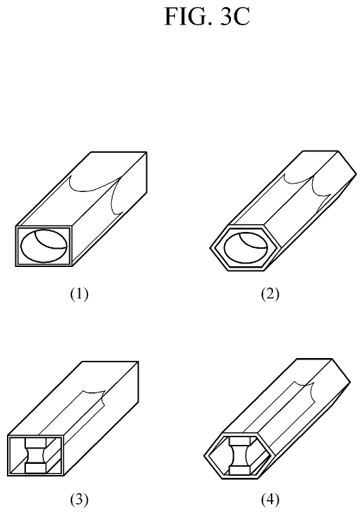

In another example, the upper passage 113 and/or the lower passage 122 may not have a constant width and/or height. In another example, the upper passage 113 may further include an auxiliary channel for facilitating capillary action. Hereinafter, a structure of the apparatus 100 for gene amplification for facilitating capillary action will be described in detail with reference to FIGS. 2A to 3C.

FIG. 2A is a front view of an apparatus for gene amplification according to an embodiment of the present disclosure. In FIG. 2A, the first inlet 111, the second inlet 112, upper passages 113a, 113b, and 113c, the first inlet connector 121, lower passages 122a, 122b, and 122c, the gene amplification chip 130, the porous medium 140, and the air pressure maintenance hole 115 are illustrated.

The upper passage may include an injection path 113a for guiding the injected sample solution and sealing solution toward the gene amplification chip 130, a main flow path 113b disposed on an upper portion of the gene amplification chip 130, and a discharge path 113c for guiding the sample solution, having passed through the main flow path 113b, and the sealing solution toward the porous medium 140.

The lower passage may include an injection path 122a for guiding the injected sealing solution toward the gene amplification chip 130, a main flow path 122b disposed on a lower portion of the gene amplification chip 130, and a discharge path 122c for guiding the sealing solution, having passed through the main flow path 122b, toward the porous medium 140.

A height hᵢ of the injection path 113a of the upper passage and a height of the injection path 122a of the lower passage may be in a range of 1 μm to 10 mm, but the heights are not limited thereto.

A height hₒ of the discharge path 113c of the upper passage and a height h_{o,u} of the discharge path 122c of the lower passage may be in a range of 1 μm to 10 mm, but the heights are not limited thereto. In this case, the height hₒ of the discharge path 113c of the upper passage and the height h_{o,u} of the discharge path 122c of the lower passage may be expressed by hᵢ−L tan α and h_{i,u}−L tan αᵤ, respectively. In this case, L denotes the length of the main flow paths 113b and 122b, and α and αᵤ denote an inclination angle of the main flow path 113b of the upper passage and an inclination of the main flow path 122b of the lower flow path, which will be described below.

The height of the main flow path 113b of the upper passage and/or the height of the main flow path 122b of the lower passage may not be constant. For example, the main flow path 113b of the upper passage and/or the main flow path 122b of the lower passage may be inclined from the injection paths 113a and 122a toward the discharge paths 113c and 122c as illustrated in FIG. 2A, but are not limited thereto.

In this case, the main flow path 113b of the upper passage may have an inclination angle α, and the main flow path 122b of the lower passage may have an inclination angle αᵤ. In this case, the inclination angles α and αᵤ may be in a range of 0° to 35°, but are not limited thereto. Further, while FIG. 2A illustrates that the inclination angles α and αᵤ are constant, but are not limited thereto, and the heights of the main flow path 113b of the upper passage and/or the main flow path 122b of the lower passage may decrease linearly. In this case, the inclination angles α and αᵤ may be different from each other.

As described above, the main flow path 113b of the upper passage and/or the main flow path 122b of the lower passage are inclined from the injection paths 113a and 122a toward the discharge paths 113c and 122c, such that due to a height difference in the injection paths and the discharge paths, the sample solution and/or the sealing solution in the main flow paths 113b and 122b may be moved smoothly. Further, as the main flow paths 113b and 122b are inclined, capillary action may easily occur in terms of Laplace pressure in a flow direction of the sample solution and/or the sealing solution, which will be described later.

FIG. 2B is a plan view of an apparatus for gene amplification according to an embodiment of the present disclosure. In FIG. 2B, the first inlet 111, the second inlet 112, an injection path 113a of the upper passage, a main flow path 113b of the upper passage, a discharge path 113c of the upper passage, and the porous medium 140 are illustrated.

Widths of the injection path 113a and/or the discharge path 113c may not be constant in a flow direction of the sample solution or the sealing solution, i.e., in a direction from the first inlet 111 to the porous medium 140.

Referring to FIG. 2B, the injection path 113a may have a width which decreases in a flow direction of the sample solution or the sealing solution, and the discharge path 113c may have a width which increases in a flow direction of the sample solution or the sealing solution. While FIG. 2B illustrates an example in which the widths of the injection path 113a and the discharge path 113c may decrease or increase linearly, but the widths are not limited thereto.

Further, FIG. 2B is a plan view in which the lower passage is not illustrated, but the injection path and the discharge path of the lower passage may have the same shapes as those of the injection path 113a and the discharge path 113c of the upper passage. However, the present disclosure is not limited thereto, and any one of the injection path 113a of the upper passage, the discharge path 113c of the upper passage, the injection path of the lower passage, and the discharge path of the lower passage, or only some thereof may have widths which are not constant in a direction toward the porous medium 140.

As the width of the injection path 113a decreases in the flow direction as illustrated in FIG. 2B, Laplace pressure in the flow direction may increase, such that a flow speed may increase by capillary action.

FIG. 3A is a plan view of an apparatus for gene amplification according to another embodiment of the present disclosure. Referring to FIG. 3A, the first inlet 111, the second inlet 112, the upper passage 113, the air pressure maintenance hole 115, and the auxiliary channel 116 may be included in the upper main body of the apparatus for gene amplification.

Herein, d₁ denotes a diameter of the first inlet, d₂ denotes a diameter of the second inlet, wᵢ denotes a width of the injection path of the upper passage, wₘ denotes a width of the main flow path of the upper passage, wₒ denotes a width of the discharge path of the upper passage, and L denotes a length of the main flow path of the upper passage.

The diameter d₁ of the first inlet 111 may be greater than the diameter d₂ of the second inlet 112. In this case, the diameter d₁ of the first inlet 111 may be greater than or equal to the width wᵢ of the injection path of the upper passage 113, and may be less than or equal to a value obtained by adding twice the width of the auxiliary channel to the width wᵢ of the injection path of the upper passage 113. The diameter d₂ of the second inlet 112 may be in a range of 0.1 μm to 4500 μm. However, the diameters d₁ and d₂ of the first inlet 111 and the second inlet 112 are not limited thereto.

The width wᵢ of the injection path of the upper passage and the width wₒ of the discharge path of the upper passage may be in a range of 1 μm to 5 mm, but are not limited thereto. The width wₘ of the main flow path of the upper passage may be in a range of 1 μm to 10 cm, but is not limited thereto. In addition, the width wₘ of the main flow path of the upper passage may increase from the injection path toward the discharge path, and then may decrease again, but is not limited thereto. The length L of the main flow path of the upper passage may be in a range of 1 μm to 10 cm, but is not limited thereto, and may vary depending on the shape of the gene amplification chip 130.

The auxiliary channel 116 may be formed on both sides of the upper passage 113. The sealing solution injected from the first inlet may move by capillary action not only through the upper passage 113 and the lower passage 122, but also through the auxiliary channel 116 formed on both sides of the upper passage 113, which will be described in detail with reference to FIGS. 3B and 3C. FIGS. 3B and 3C are diagrams illustrating a structure and an effect of the auxiliary channel.

FIG. 3B illustrates one cross-section K of FIG. 3A. FIG. 3B illustrates the gene amplification chip 130, the upper passage 130, and the auxiliary channel 116 formed on both sides of the upper passage 130, in which wᵢ and hᵢ respectively denote the width and height of the injection path of the upper passage, and a and Δh denote the width and height of the auxiliary channel, respectively.

As illustrated in FIG. 3B, the auxiliary channel 116 may be stepped with respect to the upper passage 113, i.e., may have a height difference from the upper passage 113, but is not limited thereto. For example, unlike FIG. 3B, the height hᵢ of the upper passage 113 may be equal to the height Δh of the auxiliary channel 116, or the height hᵢ of the upper passage 113 may be greater than the height Δh of the auxiliary channel 116. In this case, the width a and height Δh of the auxiliary channel 116 may be in a range of 1 μm to 5 mm, but are not limited thereto.

FIG. 3C is a diagram explaining an effect of the auxiliary channel.

In FIG. 3C, (1) and (2) are diagrams illustrating structures in which no auxiliary channel is formed, and (3) and (4) are diagrams illustrating structures in which an auxiliary channel is formed.

When all surfaces of the passage are hydrophilic, a very high Laplace pressure is generated due to a small radius of curvature of a liquid at corners thereof, such that a very fast flow is formed along the corners (Corner flow). Referring to (1) and (2) of FIG. 3C, it can be seen that a faster flow is formed along the corners of the passage. As a result, a uniform flow may not be formed in the passage, thereby generating bubbles.

This phenomenon may be resolved by providing the auxiliary channel on some of the surfaces of the passage, as illustrated in (3) and (4) of FIG. 3C. When the auxiliary channel is provided on both sides of the passage, a flow may not be formed in the corresponding direction as a driving pressure becomes zero, such that it is possible to prevent a fast flow at the corners.

That is, the auxiliary channel may serve to allow the sample solution, having a limited volume, to continuously move by capillary action toward the porous medium through the upper passage without external power.

The lower main body of the apparatus for gene amplification may not include the auxiliary channel.

Referring back to FIG. 1, the sample solution may flow through the upper passage 113 to be loaded into the gene amplification chip 130, in which case the gene contained in the sample solution may be amplified.

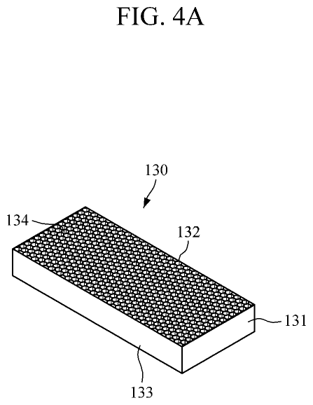

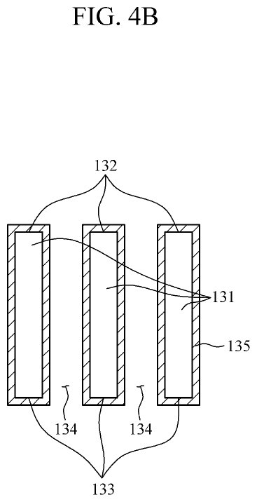

The gene amplification chip 130 may be inserted between the upper main body 110 and the lower main body 120. For example, the gene amplification chip 130 may be inserted into an insertion groove (e.g., insertion groove 123) of the gene amplification chip, which may be included in the upper main body 110 and the lower main body 120. A shape of the gene amplification chip 130 and the photothermal film disposed on the gene amplification chip 130 will be described below with reference to FIGS. 4A and 4b.

FIG. 4A is a diagram illustrating a gene amplification chip according to an embodiment of the present disclosure.

Referring to FIG. 4A, the gene amplification chip 130 includes a substrate 131, a substrate upper surface 132, a substrate lower surface 133, and an array of through holes 134.

The substrate 131 may be made of any one of inorganic matter, such as silicon (Si), glass, polymer, metal, ceramic, graphite, etc., and acrylic material, polyethylene terephthalate (PET), polycarbonate, polystylene, and polypropylene, but is not limited thereto.

In this case, the substrate 131 may be made of a hydrophilic material having a contact angle of 90° or less with respect to water, but is not limited thereto.

A thickness of the substrate, i.e., a length from the upper surface 132 to the lower surface 133 of the substrate 131, may be 1 mm or less, but is not limited thereto, and may be changed to various numbers.

As illustrated herein, the through holes 134 may pass through the substrate 131 in a direction from the upper surface 132 to the lower surface 133. When the through holes 134 are formed, an etching process such as Deep Reactive Ion Etching (DRIE) or a thinning process including CMP treatment may be performed. A volume of the through holes 134 may be 1 nL or less, and the number of the through holes 134 may be at least 20,000 or more. The through holes 134 may have a cylindrical or hexagonal prism shape, but its shape is not limited thereto, and may be formed in various shapes such as other polygonal prism and the like. In the case where the through holes 134 have the hexagonal prism shape, a diagonal distance of a cross-section of the through holes 134 may be 100 μm or less. However, the number, shape, and volume of the through holes 134 are not limited thereto, and may be changed variously.

A gene amplification reaction occurs in the through holes 134. In this case, reverse transcription of an RNA sample is performed in the respective through holes 134 by using a reverse transcriptase. The gene amplification reaction may include, for example, a nucleic acid amplification reaction including at least one of polymerase chain reaction (PCR) amplification and isothermal amplification, a redox reaction, a hydrolytic reaction, and the like. In this case, the gene may include a duplex of one or more of ribonucleic acid (RNA), deoxyribonucleic acid (DNA), peptide nucleic acid (PNA), and locked nucleic acid (LNA), but is not limited thereto. While the gene amplification reaction is performed in the through holes 134, an optical signal is measured by the optical unit and/or the processor of the apparatus for gene amplification, and the amplified gene may be detected based on the measured optical signal. In this case, the optical signal may include fluorescence, phosphor, absorbance, surface plasmon resonance, and the like. As described above, the gene amplification chip 130 may be used to detect, for example, the presence of a target DNA template, quantitative information, and the like.

A structure for removing gas bubbles, e.g., a bubble trap or a bubble removing member/chamber, and/or a gas permeable material, etc., may be disposed in the respective through holes 134 or at the inlet of the array of the through holes 134.

The gene amplification chip 130 may include an optical heating element, such as a photothermal film, which reacts to an external light source. However, the gene amplification chip 130 is not limited thereto, and may also include an electrical heating element, such as a Peltier element and the like, to have electrothermal properties, instead of the optical heating element. For convenience of explanation, a shape of the gene amplification chip 130, on which the photothermal film as an example of the optical heating element is deposited, will be described below with reference to FIG. 4B.

FIG. 4B is a diagram illustrating a side surface of the gene amplification chip 130, on which a photothermal film is deposited.

Referring to FIG. 4B, the gene amplification chip 130 may further include a photothermal film 135 in addition to the substrate 131, the substrate upper surface 132, the substrate lower surface 133, and the array of the through holes 134 which are described above. FIG. 4B illustrates a state in which the photothermal film 135 is deposited on the substrate upper surface 132, the substrate lower surface, and a partition wall of the through holes 134. In this case, the photothermal film may be deposited in a pattern.

Unlike the example of FIG. 4B, however, the photothermal film 135 may be deposited on only any one of the substrate upper surface 132, the substrate lower surface 133, and the partition wall of the through holes 134, or may be deposited on only the substrate upper surface 132 and the substrate lower surface 133, which is more desirable in terms of process complexity or production costs than the case where the photothermal film 135 is deposited on all of the substrate upper surface 132, the substrate lower surface, and the partition wall of the through holes 134.

A thickness of the photothermal film 135 may be 10 μm or less, but is not limited thereto. Further, the photothermal film 135 may be formed as a metal layer, but is not limited thereto and may be made of a metal oxide material, metalloid, and base metal. For example, the photothermal film 135 may be formed of a tungsten-based material having excellent infrared absorptivity, and thus achieving a photothermal conversion effect during laser emission. The photothermal film 135 may have a nanostructure. For example, the photothermal film 135 may be formed as nanoparticles, nanorod, nanodisc, or nanoisland, which has a size of 50 nm or less in diameter and 50 nm or less in thickness, but is not limited thereto, and may be formed in various nanostructures.

Further, although not illustrated in FIG. 4B, the photothermal film 135 may further contain carbon black, visible light dye, ultraviolet dye, infrared dye, fluorescent dye, radiation-polarizing dye, pigment, metallic compound, and another suitable absorber material as a photothermal conversion material.

The photothermal film 135 may receive light from a light source, and may generate heat by photonic heating using the received light. In this case, as the photothermal film 135 is disposed at a plurality of positions of the gene amplification chip 130, temperature may be controlled at a uniform level, and heat generation efficiency may be improved.

In addition to the photothermal film 135, the gene amplification chip 130 may further include: an adhesive layer disposed between the substrate 131 and the photothermal film 135 and improving adhesive strength of the photothermal film 135; a separate element for improving adhesive strength between the photothermal film 135 and the substrate 131; an auxiliary film disposed to surround the photothermal film 135 and preventing hindrance to the gene amplification process within the through holes; and other material for amplifying the photothermal effect of the photothermal film 135.

Referring back to FIG. 1, the porous medium 140 may be inserted between the upper main body 110 and the lower main body 120. For example, the porous medium 140 may be inserted into an insertion groove (e.g., insertion groove 124) of the porous medium, which may be included in the upper main body 110 and the lower main body 120, as described above.

The porous medium 140 may be made of a hydrophilic material. For example, the porous medium 140 may be formed of cotton, filter paper, hydrogel, sponge, and the like

The porous medium 140 may include a plurality of pores or a plurality of pin type microstructures. In this case, a diameter of the pores or the pin type microstructures is smaller than the widths of the upper passage and the lower passage, and may be in a range of 0.001 μm to 100 μm, but is not limited thereto. A distance between the plurality of pores or a distance between the plurality of the pin type microstructures may be in a range of 0.001 μm to 100 μm, but is not limited thereto.

The porous medium 140, which has a wide surface area for its volume with high absorbing properties, may pull the sample solution with a greater force than the upper passage 113, thereby absorbing the sample solution rapidly. In this case, the porous medium 140 may absorb a large amount of sample solution and sealing solution with a limited length, thereby allowing the apparatus 100 for gene amplification to be manufactured in a smaller size.

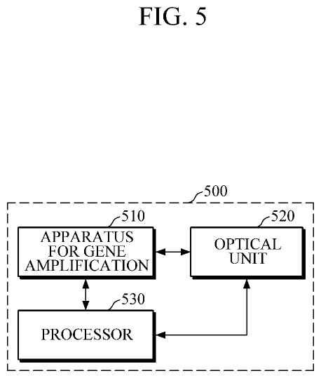

FIGS. 5 to 8 are block diagrams illustrating an apparatus for detecting a microfluid according to embodiments of the present disclosure.

Referring to FIG. 5, an apparatus 500 for detecting a microfluidic includes an apparatus 510 for gene amplification, an optical unit 520, and a processor 530. The apparatus 510 for gene amplification is described in detail above with reference to FIGS. 1 to 4B, such that the following description will be focused on the optical unit 520 and the processor 530.

The optical unit 520 may measure an optical signal while the gene amplification reaction is performed in the respective through holes of an array of micro/nano through holes. In this case, the optical signal may include fluorescence, phosphor, absorbance, surface plasmon resonance, and the like. The optical unit 520 may include a light source for emitting light onto the sample solution in the micro/nano through holes, and a detector (e.g., a light detector such as a photodiode) for detecting the optical signal reflected from the sample solution in the micro/nano through holes. The light source may include LED, laser, vertical-cavity surface-emitting laser (VCSEL), etc., but is not limited thereto. Further, the detector may include a photomultiplier tube, a photo detector, a photomultiplier tube array, a photo detector array, a complementary metal-oxide semiconductor (CMOS) image sensor, etc., but is not limited thereto. In addition, the optical unit 520 may further include a filter for passing light of a specific wavelength, a mirror for directing the light radiating from the micro/nano through holes toward the detector, a lens for collecting light radiating from the micro/nano through holes, and the like.

The processor 530 may be electrically connected to the optical unit 520, and may control driving of the light source of the optical unit 520. Further, the processor 530 may receive the optical signal from the detector and analyze the optical signal, and may detect biomolecules based on the analysis. For example, the processor 530 may perform quantitative analysis of the amplified gene based on a result of digital nucleic acid amplification, detected by the detector, and Poisson distribution.

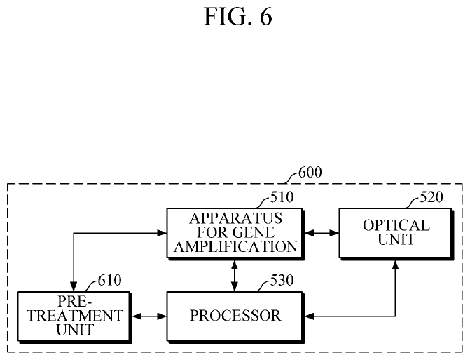

Referring to FIG. 6, an apparatus 600 for detecting a microfluid according to an embodiment of the present disclosure may further include a pre-treatment unit 610 in addition to the configuration of the apparatus 500 for detecting a microfluid of FIG. 5.

The pre-treatment unit 610 may perform a pre-treatment process, such as heating the sample solution present in the main flow path of the upper passage, chemical treatment, treatment with magnetic beads, solid phase extraction, treatment with ultrasonic waves, and the like. To this end, the pre-treatment unit 610 may include various materials or structures for pre-treatment, such as magnetic beads, an ultrasonic device, an optical/electric heating device, etc., which are provided inside and/or outside of the main flow path of the upper passage, and the pre-treatment unit 610 may control these materials or structures. At least some of the functions of the pre-treatment unit 610 may be integrated into the processor 530.

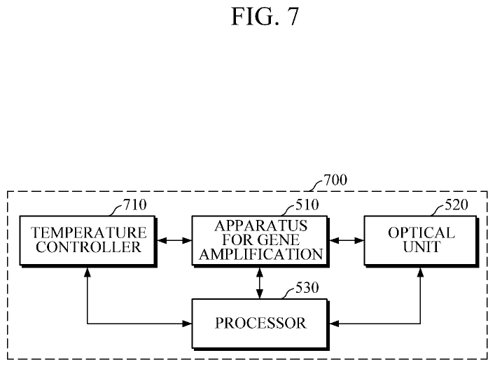

Referring to FIG. 7, an apparatus 700 for detecting a microfluid according to an embodiment of the present disclosure may further include a temperature controller (e.g., a thermostat, a heating system, and/or a cooling system) 710 in addition to the apparatus 500 or 600 for detecting a microfluid according to the embodiment of FIG. 5 or FIG. 6.

The temperature controller 710 may control temperature of the sample solution present in in the upper passage and/or the respective through holes of the gene amplification chip.

For example, the temperature controller 710 may control temperature of the sample solution present in the injection path of the upper passage to be maintained at an isothermal temperature of 95° C. or higher, or to be maintained at an isothermal temperature within a range of 30° C. to 60° C.

In another example, the temperature controller 710 may control temperature of the sample solution loaded into the gene amplification chip to be, for example, a thermal dissolution temperature, a reverse transcription temperature and a gene amplification temperature.

In this case, the temperature controller 710 may include an electric heater and/or an optical heater, and may control the temperature of the sample solution by using the electric heater and/or optical heater.

The electric heater may include, for example, a heating element and/or a Peltier element. The optical heater may include, for example, one or more light sources disposed outside of the apparatus 510 for gene amplification and emitting light onto the gene amplification chip included in the apparatus 510 for gene amplification, and the like.

Further, the temperature controller 710 may include a temperature sensor disposed inside or outside of the apparatus 510 for gene amplification and measuring the temperature of the sample solution present in the upper passage and the gene amplification chip. In this case, the temperature sensor may include a thermocouple having a bimetal junction generating temperature-dependent electric and magnetic fields (EMFs), a resistive thermometer including materials having electrical resistance proportional to temperature, thermistors, an integrated circuit (IC) temperature sensor, a quartz thermometer, etc., but is not limited thereto.

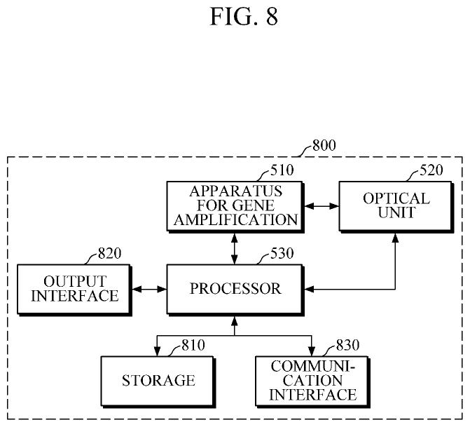

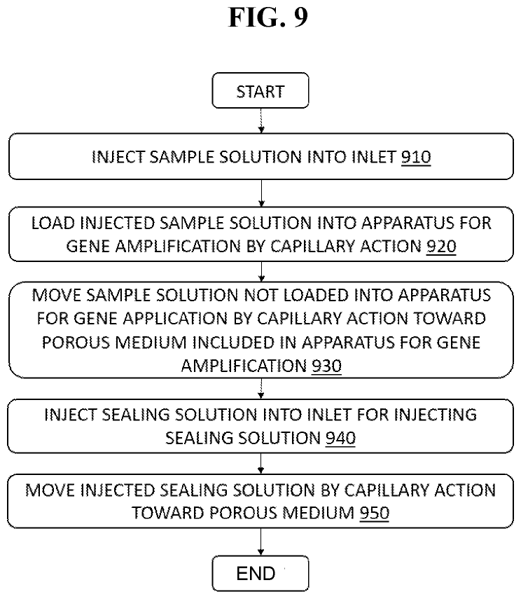

Referring to FIG. 8, an apparatus 800 for detecting a microfluid according to an embodiment of the present disclosure may further include a storage 810, an output interface 820, and a communication interface 830 in addition to the configuration of the apparatus 500, 600, and 700 for detecting a microfluid according to the embodiment of FIG. 7, FIG. 8, or FIG. 9.

The storage 810 may store, for example, a variety of reference information for gene amplification and/or a gene amplification result, and the like. The storage 810 may include at least one storage medium of a flash memory type memory, a hard disk type memory, a multimedia card micro type memory, a card type memory (e.g., an SD memory, an XD memory, etc.), a Random Access Memory (RAM), a Static Random Access Memory (SRAM), a Read Only Memory (ROM), an Electrically Erasable Programmable Read Only Memory (EEPROM), a Programmable Read Only Memory (PROM), a magnetic memory, a magnetic disk, and an optical disk, and the like, but is not limited thereto.

The output interface 820 may output, for example, a gene amplification process, a gene amplification result and/or interaction information with a user during the gene amplification process, and the like. The output interface 820 may provide a user with information by visual, audio, and tactile methods and the like using a visual output module (e.g. display), an audio output module (e.g., speaker), a haptic module, and the like.

The communication interface 830 may communicate with an external device. For example, the communication interface 830 may transmit data generated by the apparatus 800 for detecting a microfluid, e.g., a gene amplification result, and the like to an external device, and may receive data required for gene amplification and/or for analysis of the gene amplification result from the external device. In this case, the external device may be medical equipment, a printer to print out results, or a display device. In addition, the external device may be a digital TV, a desktop computer, a mobile phone, a smartphone, a tablet PC, a laptop computer, Personal Digital Assistants (PDA), Portable Multimedia Player (PMP), a navigation device, an MP3 player, a digital camera, a wearable device, etc., but is not limited thereto.

The communication interface 830 may communicate with the external device by using Bluetooth communication, Bluetooth Low Energy (BLE) communication, Near Field Communication (NFC), WLAN communication, Zigbee communication, Infrared Data Association (IrDA) communication, Wi-Fi Direct (WFD) communication, Ultra-Wideband (UWB) communication, Ant+ communication, WIFI communication, Radio Frequency Identification (RFID) communication, 3G, 4G, and 5G communications, and the like. However, this is merely exemplary and is not intended to be limiting.

FIG. 9 is a flowchart illustrating a method of gene amplification according to an embodiment of the present disclosure.

The method of gene amplification of FIG. 9 may be performed by the apparatuses 500, 600, 700, and 800 for detecting a microfluid according to the embodiments of FIGS. 5 to 8, which are described in detail above, and thus will be briefly described below in order to avoid redundancy.

First, the apparatus for detecting a microfluid may inject a sample solution into an inlet for injecting the sample solution in operation 910.

Then, the injected sample solution may be loaded into the gene amplification chip by capillary action in operation 920.

The injected sample solution may move by capillary action through the upper passage. In this case, the upper passage may have a material and structure for facilitating capillary action. For example, the upper passage may be made of a material having a contact angle of 10° or less with respect to water. In another example, the main flow path of the upper passage may be inclined from the injection path toward the discharge path, and/or widths of the injection path and the discharge path may not be constant in a flow direction of the sample solution. A detailed description thereof will be omitted.

Then, the sample solution not loaded into the gene amplification chip may move by capillary action toward the porous medium included in the apparatus for gene amplification in operation 930.

In this case, capillary action may easily occur with a material and structure for facilitating capillary action, and/or high hydrophilic properties of the porous medium. A detailed description thereof will be omitted.

Subsequently, the apparatus for detecting a microfluid may inject a sealing solution into an inlet for injecting the sealing solution in operation 940.

In this case, a diameter of the inlet, into which the sealing solution is injected, may be greater than a diameter of the inlet into which the sample solution is injected. The sealing solution may be a non-polar solution that is not mixed with the sample solution. When a gene amplification reaction occurs with the sample solution being loaded into the gene amplification chip, if an upper surface and a lower surface of the gene amplification chip are in contact with a gas, the sample solution may be evaporated and lost rapidly during the gene amplification process. In this case, the sealing solution may be coated on the upper surface, the lower surface, and the like of the gene amplification chip, thereby preventing loss of the loaded sample solution. A detailed description thereof will be omitted.

Then, the injected sealing solution may move by capillary action toward the porous medium in operation 950. In this case, the sealing solution moves through the upper passage, the lower passage, and the auxiliary channel formed on both sides of the upper passage, to be coated on the upper surface, the lower surface, and the like of the gene amplification chip, thereby preventing loss of the loaded sample solution. A detailed description thereof will be omitted.

Subsequently, while the gene amplification reaction is performed in the gene amplification chip, or after the gene amplification reaction is complete, the apparatus for detecting a microfluid may measure an optical signal from the sample solution, and may perform quantitative analysis of the amplified gene by using the measured optical signal. In this case, by emitting light of a predetermined wavelength onto the gene amplification chip using the light source of the optical unit for a predetermined period of time, the apparatus for detecting a microfluid may detect an optical signal, such as fluorescence, phosphorescence, absorbance, surface plasmon resonance, etc., radiating from the sample of the gene amplification chip, and may perform quantitative analysis of the amplified gene based on the detected optical signal and Poisson distribution. A detailed description thereof will be omitted.

The present invention can be realized as a computer-readable code written on a computer-readable recording medium. The computer-readable recording medium may be any type of recording device in which data is stored in a computer-readable manner.

Examples of the computer-readable recording medium include a ROM, a RAM, a CD-ROM, a magnetic tape, a floppy disc, an optical data storage, and a carrier wave (e.g., data transmission through the Internet). The computer-readable recording medium can be distributed over a plurality of computer systems connected to a network so that a computer-readable code is written thereto and executed therefrom in a decentralized manner. Functional programs, codes, and code segments needed for realizing the present invention can be easily deduced by computer programmers of ordinary skill in the art, to which the present invention pertains.

The foregoing exemplary embodiments are merely exemplary and are not to be construed as limiting. The present teaching can be readily applied to other types of apparatuses. Also, the description of the exemplary embodiments is intended to be illustrative, and not to limit the scope of the claims, and many alternatives, modifications, and variations will be apparent to those skilled in the art.

What is claimed is:

1. An apparatus for gene amplification, the apparatus comprising:
an upper main body comprising a first inlet to receive a sealing solution, a second inlet to receive a sample solution, and an upper passage that allows the sample solution and the sealing solution to move by capillary action;
a lower main body disposed to oppose the upper main body, and comprising a lower passage through which the sealing solution moves by capillary action after being injected from the first inlet of the upper main body;
a gene amplification chip configured to be inserted between the upper main body and the lower main body; and
a porous medium configured to be inserted between the upper main body and the lower main body.

2. The apparatus of claim 1, wherein:
the upper passage comprises a first injection path for guiding the sample solution and the sealing solution toward the gene amplification chip, a first main flow path disposed on an upper portion of the gene amplification chip, and a first discharge path for guiding the sample solution and the sealing solution toward the porous medium; and
the lower passage has a second injection path for guiding the sealing solution toward the gene amplification chip, a second main flow path disposed on a lower portion of the gene amplification chip, and a second discharge path for guiding the sealing solution toward the porous medium.

3. The apparatus of claim 2, wherein the first main flow path is inclined from the first injection path toward the first discharge path, or the second main flow path is inclined from the second injection path toward the second discharge path.

4. The apparatus of claim 3, wherein at least one of the first main flow path and the second main flow path has an inclination angle of 0° to 35°.

5. The apparatus of claim 2, wherein at least one of the first injection path, the first discharge path, the second injection path, and the second discharge path has a width which is not constant in a flow direction of the sample solution or the sealing solution.

6. The apparatus of claim 5, wherein a width of the first injection path and a width of the second injection path linearly decrease in the flow direction of the sample solution or the sealing solution.

7. The apparatus of claim 5, wherein a width of the first discharge path and a width of the second discharge path linearly increase in the flow direction of the sample solution or the sealing solution.

8. The apparatus of claim 2, wherein:
a width of the first injection path, a width of the second injection path, a width of the first discharge path, and a width of the second discharge path are in a range of 1 μm to 5 mm; and
a width of the first main flow path and a width of the second main flow path are in a range of 1 μm to 10 cm.

9. The apparatus of claim 1, wherein the upper main body further comprises an auxiliary channel which is provided on both sides of the upper passage, and which allows the sealing solution to move by capillary action through the auxiliary channel.

10. The apparatus of claim 9, wherein the auxiliary channel is stepped with respect to the upper passage.

11. The apparatus of claim 1, wherein the upper passage and the lower passage comprise a hydrophilic material having a contact angle of 90° or less with respect to water.

12. The apparatus of claim 1, wherein the sealing solution is a non-polar solution that is not mixed with the sample solution.

13. The apparatus of claim 1, wherein the porous medium comprises a hydrophilic material, and has a plurality of pores or a plurality of pin type microstructures.

14. The apparatus of claim 13, wherein:
a diameter of each of the plurality of pores or each of the plurality of pin type microstructures is in a range of 0.001 μm to 100 μm, and is smaller than a width of the upper passage and a width of the lower passage; and
a distance between the plurality of pores or a distance between the plurality of pin type microstructures is in a range of 0.001 μm to 100 μm.

15. The apparatus of claim 1, wherein a diameter of the first inlet is greater than a diameter of the second inlet.

16. The apparatus of claim 15, wherein:
the diameter of the first inlet is greater than or equal to a width of an injection path of the upper passage; and
the diameter of the second inlet is in a range of 0.1 μm to 4500 μm.

17. The apparatus of claim 1, wherein the upper main body further comprises an air pressure maintenance hole disposed on an upper portion of the porous medium.

18. The apparatus of claim 1, wherein the gene amplification chip comprises a substrate, and an array of through holes which pass through the substrate in a direction from an upper surface to a lower surface of the substrate, and in which a gene amplification reaction occurs.

19. The apparatus of claim 18, wherein the gene amplification chip comprises a photothermal film disposed on at least one of the upper surface and the lower surface of the substrate, and a partition wall of the respective through holes, and generating heat by using received light.

20. An apparatus for detecting a microfluid, the apparatus comprising:
a gene amplifier;
an optical unit comprising a light emitter and a light detector to emit light onto a sample solution and to measure an optical signal scattered or reflected from the sample solution, while a gene amplification reaction is performed in a gene amplification chip of the gene amplifier, or after the gene amplification reaction is complete; and
a processor configured to detect an amplified gene by analyzing the optical signal,
wherein the gene amplifier comprises:
an upper main body comprising a first inlet to receive a sealing solution, a second inlet to receive the sample solution, and an upper passage that allows the sample solution and the sealing solution to move by capillary action;
a lower main body disposed to oppose the upper main body, and having a lower passage through which the sealing solution moves by capillary action after being injected from the first inlet of the upper main body;
the gene amplification chip configured to be inserted between the upper main body and the lower main body; and
a porous medium configured to be inserted between the upper main body and the lower main body.
